(19) United States
(12) Patent Application Publication
(10) Pub. No.: US 2026/0345212 A1
(43) Pub. Date: Nov. 5, 2026

(54) BONE SCREW AND METHOD OF MANUFACTURING SAME

(71) Applicant: Eminent Spine LLC, Plano, TX (US)

(72) Inventor: Steve Courtney, Plano, TX (US)

(21) Appl. No.: 19/666,209

(22) Filed: May 2, 2026

Related U.S. Application Data
(60) Provisional application No. 63/798,685, filed on May 2, 2025.

(51) Int. Cl. A61B 17/86

(52) U.S. Cl. CPC A61B 17/864; A61B 17/863; A61B 17/866

(57) ABSTRACT

A bone screw and methods of manufacture are disclosed. The screw has first, second, and third helical threads spanning a cancellous segment with a wider pitch and a cortical segment with a tighter pitch, offset by about 120° for a triple-lead configuration. This speeds insertion while ensuring robust fixation. An axial bore with at least two fenestrations allows targeted delivery of biologics or cement. An osteo-porous lattice, about 10%–20% porous and resembling spongy bone, is formed on the shank to foster bone ingrowth and minimize stress shielding. The entire screw, including this lattice, is made in a single additive build from a titanium alloy (e.g., 6Al–4V ELI). Post-processing, such as hot isostatic pressing, optimizes mechanical properties. A driver-engagement head and a self-tapping flute-defined tip further enhance deployment. This design is highly efficient and biocompatible, suitable for varied bone densities and spinal fusion applications.

### PRIORITY CLAIM

This application claims priority to US Application Serial No. 63/798,685 entitled “Bone Screw and Method of Manufacturing Same” and filed on May 2, 2025 in the name of Steve Courtney, which is hereby incorporated by reference, in entirety, for all purposes.

### TECHNICAL FIELD OF THE INVENTION

The present disclosure concerns orthopedic bone fasteners, especially bone screws used to anchor implants or devices to skeletal structures. More specifically, it focuses on bone screw designs that enhance fixation strength, streamline insertion, and reduce surgical complexity in spine surgery.

### BACKGROUND OF THE INVENTION

Bone screws are widely employed in orthopedic and spine surgery to stabilize fractures, fix implants, and correct skeletal deformities. In spinal fusion procedures, for example, pedicle screws must endure complex loads—compressive, distractive, and torsional—arising from normal patient movements. Conventional designs sometimes exhibit suboptimal pull-out strength or risk loosening over time, potentially leading to screw back-out, hardware failure, or revision surgery. Surgeons thus face significant challenges in maintaining reliable fixation, especially in higher-stress environments like the lumbar spine or in osteoporotic bone.

Efforts to enhance bone screws have included modifications to thread geometry, self-tapping features, and fenestrations that allow biologics to be delivered near the implant site. However, traditional manufacturing methods often limit the ability to integrate advanced design elements—such as gradient porosities or complex multi-thread profiles—into a single device. Consequently, many existing screws cannot combine efficient insertion, robust mechanical properties, osteo-integration, and multi-density adaptability within a single construct.

As minimally invasive techniques become more common and operating times are pressured to decrease, the demand grows for bone screws offering quick insertion, secure fixation, and compatibility with various bone densities. Additive manufacturing processes provide new opportunities for creating lattice structures, embedded fenestrations, and specialized threads that distribute load more evenly, facilitate bone on-growth, and reduce insertion torque. These developments underscore the need for next-generation orthopedic screws capable of delivering improved surgical outcomes under diverse clinical requirements.

### SUMMARY OF THE INVENTION

To address these challenges, the present disclosure introduces a bone screw that consolidates multiple performance-enhancing features into one integrated solution. Its shank is configured with a triple-lead thread arrangement—first, second, and third helical threads offset by 120°—that expedites insertion while maintaining high pull-out strength. Each thread extends across two axial segments: a cancellous segment with a more widely spaced pitch and a cortical segment with a tighter pitch, enabling the screw to engage varying bone densities within a single vertebra.

An axial bore, coupled with at least two fenestrations, allows targeted delivery of bone cement, growth factors, or other agents, improving fixation in osteoporotic bone and promoting faster healing. The screw also features an osteo-porous lattice structure on at least part of the shank, formed by additive manufacturing with about 10%–20% porosity. This lattice may approximate a scanned or modeled three-dimensional trabecular or cancellous bone morphology, including a morphology derived from CT data, micro-CT data, laser-scanning data, CAD-defined trabecular geometry, a mathematical trabecular-bone model, or combinations thereof, thereby promoting bone ingrowth, mitigating stress shielding, and reducing the likelihood of loosening under repetitive loading. Produced in a single additive build from a titanium alloy (e.g., 6Al–4V ELI), the bone screw may undergo hot isostatic pressing to further optimize material density and fatigue strength. A specialized driver interface on the head ensures consistent torque application, while a self-tapping flute at the distal tip simplifies insertion. By uniting multi-thread geometry, an osteo-porous scaffold, and fenestration-based biologic delivery, this design enhances mechanical performance, reduces procedure time, and improves overall patient outcomes—offering surgeons a comprehensive tool for diverse orthopedic and spinal applications.

In certain embodiments, each of the first, second, and third helical threads is a continuous thread start extending through both the cancellous thread form and the cortical thread form while maintaining the approximately 120° circumferential phase offset along the shank, including through a transition region between the cancellous and cortical thread forms. The thread crest portions of the first, second, and third helical threads may be formed as substantially fully dense titanium alloy load-bearing regions, while the osteo-porous lattice structure is disposed primarily or exclusively in thread flank regions, inter-thread root regions, and/or regions adjacent the minor diameter of the shank. In this manner, the screw preserves dense, mechanically strong thread crests for insertion and pull-out resistance while providing porous regions positioned to promote bone ingrowth and material distribution.

In some embodiments, the fenestrations open from the axial bore into inter-thread root regions occupied by or adjacent to the osteo-porous lattice structure. The fenestrations may be circumferentially positioned between adjacent ones of the first, second, and third helical threads so that the fenestrations do not intersect the load-bearing thread crests. In one preferred embodiment, the fenestrations are located in the cancellous thread form or distal cancellous segment, and the cortical thread form or proximal cortical segment is substantially free of fenestrations.

### BRIEF DESCRIPTION OF THE DRAWINGS

For a more complete understanding of the features and advantages of the present invention, reference is now made to the following detailed description in conjunction with the accompanying figures, wherein corresponding numerals refer to corresponding parts throughout:

FIG. 1 is a proximal-end perspective view of an embodiment of a bone screw.

FIG. 2 is a distal-end perspective view of the bone screw of FIG. 1.

FIG. 3 is a side elevation view of the bone screw of FIG. 1.

FIG. 4 is a proximal-end view of the bone screw of FIG. 1.

FIG. 5 is a distal-end view of the bone screw of FIG. 1.

FIG. 6 is a longitudinal cross-sectional view of the bone screw of FIG. 1.

FIG. 7 is an enlarged, detailed view illustrating an osteo-porous lattice structure formed on the bone screw.

FIG. 8 is an enlarged, detailed view illustrating another osteo-porous lattice structure formed on the bone screw.

FIG. 9 is a flowchart illustrating an example method of manufacturing the bone screw.

FIG. 10 is a perspective, exploded view of the bone screw shown in FIG. 1, depicted as part of a bone screw fixation system.

FIG. 11 is a side elevation view of the bone screw shown in FIG. 1, implemented with an anterior buttress staple.

FIG. 12 is a front elevation view of the bone screw shown in FIG. 1 with a pedicle screw arrangement.

FIG. 13 is a front elevation view of the bone screw shown in FIG. 1 with a further pedicle screw arrangement.

### DETAILED DESCRIPTION OF THE INVENTION

While the making and using of various embodiments of the present invention are discussed in detail below, it should be appreciated that the present invention provides many applicable inventive concepts, which can be embodied in a wide variety of specific contexts. The specific embodiments discussed herein are merely illustrative of specific ways to make and use the invention, and do not delimit the scope of the present invention.

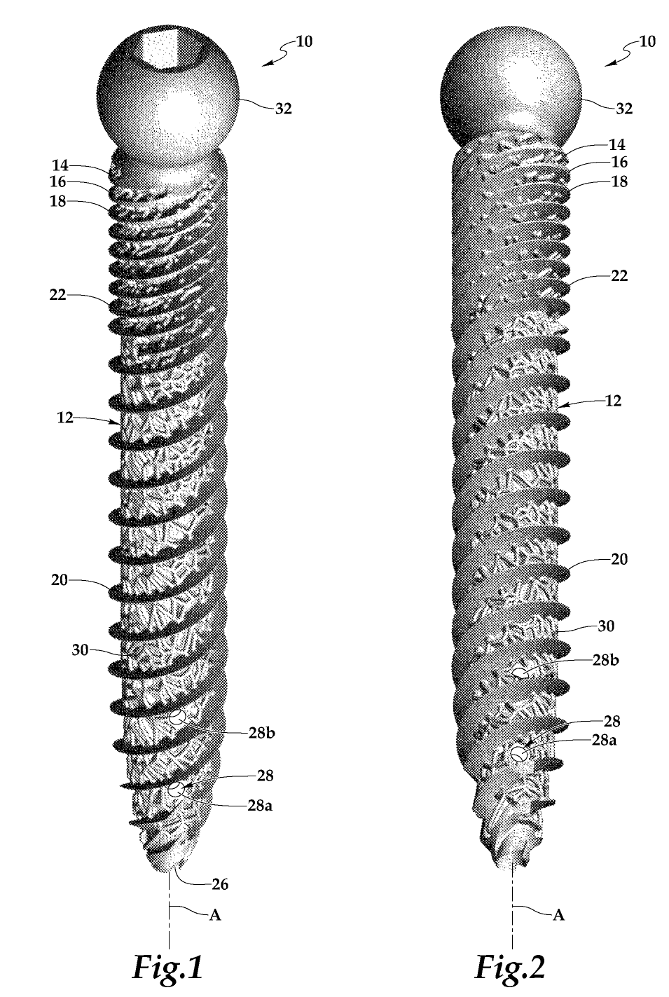

Referring initially to FIGS. 1 through 6, therein is depicted one embodiment of a bone screw 10. With respect to FIG. 1, a proximal-end perspective view illustrates one embodiment of the bone screw 10 configured for use in orthopedic and spinal fusion procedures. The bone screw 10 features a shank 12 through which first, second, and third helical threads 14, 16, 18 extend. Each of these three threads traverses two distinct axial segments along the shank 12: a cancellous thread form 20 having a more widely spaced pitch for engaging trabecular bone, and a cortical thread form 22 with a tighter pitch for anchoring in denser cortical regions. The leads of threads 14, 16, 18 are offset by approximately 120° relative to one another, creating a triple-lead configuration that expedites insertion while preserving robust mechanical fixation. An axial bore 26 runs from the proximal end of the bone screw 10 to the distal end along an axis A, intersected by at least two fenestrations 28a, 28b, which are collectively fenestrations 28. Each fenestration 28 intersects the axial bore 26 in certain embodiments. These fenestrations 28 facilitate the injection of biologic or cementitious materials—an especially valuable feature in osteoporotic bone scenarios or to promote spinal fusion. The bone screw 10 further comprises an osteo-porous lattice structure 30, formed on at least part of the shank 12 during an additive manufacturing process. This osteo-porous lattice structure 30 may exhibit a porosity of approximately 10%–20% and may mimic, approximate, or be generated from a scanned or modeled three-dimensional trabecular or cancellous bone morphology, encouraging bone ingrowth and reducing stress shielding. Notably, the entire bone screw 10, including its osteo-porous lattice structure 30, is produced in a single build from a titanium alloy, for example 6Al–4V ELI, thereby avoiding seams or bonding interfaces common in traditional manufacturing approaches.

Turning to FIG. 2, a distal-end perspective view highlights how the cancellous thread form 20 occupies a more distally oriented segment of the shank 12, incorporating a wider pitch dimension—such as a major pitch near 2.67 mm (0.105 in.) and a minor pitch near 1.34 mm (0.053 in.)—and a thread thickness between roughly 0.26 mm (0.010 in.) and 0.18 mm (0.007 in.). Meanwhile, the cortical thread form 22 is found more proximally, featuring a tighter pitch for gripping firmer bone near the cortical shell. Both thread forms share a common flank angle of about 36° and an included angle of about 60°, helping maintain consistent engagement across varying bone densities. In another implementation, both thread forms may have an included thread angle of about 60°, as measured in an axial cross-section between opposed flank surfaces of a thread. In addition, each of the first, second, and third helical threads 14, 16, 18 may have a lead angle or helix angle of about 36°, as measured at a selected pitch diameter between a line tangent to the helical thread path and a plane perpendicular to the longitudinal axis A of the shank 12. As used herein, the lead angle or helix angle is distinct from the included thread angle; therefore, the lead angle or helix angle need not be one half of the included thread angle. The three helical threads 14, 16, 18 each begin near the proximal portion of the shank 12 and continue toward a distal tip 36, carrying their respective leads in a triple-lead arrangement. This geometry provides a faster advancement rate—approximately three times greater per revolution—compared to single-lead designs, thus minimizing insertion time and torque demands.

For purposes of this disclosure, each of the first, second, and third helical threads 14, 16, 18 may be considered a separate thread start. Each thread start may be continuous from a proximal region of the shank 12 through the cortical thread form 22, through a transition region, and through the cancellous thread form 20 toward the distal tip 36. The cancellous thread form 20 and cortical thread form 22 therefore need not be separate thread members; rather, in certain embodiments, they are different pitch regions of the same three continuous thread starts. The approximately 120° circumferential offset between the first, second, and third helical threads 14, 16, 18 may be maintained along the shank 12, including through the transition between the cortical thread form 22 and the cancellous thread form 20.

Each helical thread 14, 16, 18 may include an outer crest portion 43, opposed flank regions 45, 47 and an inter-thread root or valley region 49 located between adjacent crest portions. The outer crest portions 43 are configured as primary load-bearing surfaces that engage bone during insertion and resist pull-out. The flank regions 45, 47 and inter-thread root regions 49 may be positioned closer to the minor diameter of the shank 12 and may provide locations for the osteo-porous lattice structure 30. As used herein with respect to the externally threaded shank 12, the “major diameter” is the diameter measured across the radially outermost crest portions of the helical threads 14, 16, 18, and the “minor diameter” or “root diameter” is the diameter measured across the inter-thread root or valley regions between adjacent thread crests. Thus, for example, regions “near the minor diameter” are regions adjacent the threaded core of the shank 12, including root regions and adjacent flank regions, rather than the radially outermost load-bearing crest portions. As used herein, the “included thread angle” refers to the angle between opposed flank surfaces of a thread when viewed in an axial cross-section taken through the longitudinal axis A of the shank 12. By contrast, the “lead angle” or “helix angle” refers to the angular orientation of the helical path of a thread around the shank 12, and may be measured at a selected pitch diameter relative to a plane perpendicular to the longitudinal axis A. Accordingly, an included thread angle of about 60° and a lead angle or helix angle of about 36° describe different geometric relationships of the thread and are not intended to define the same angle.

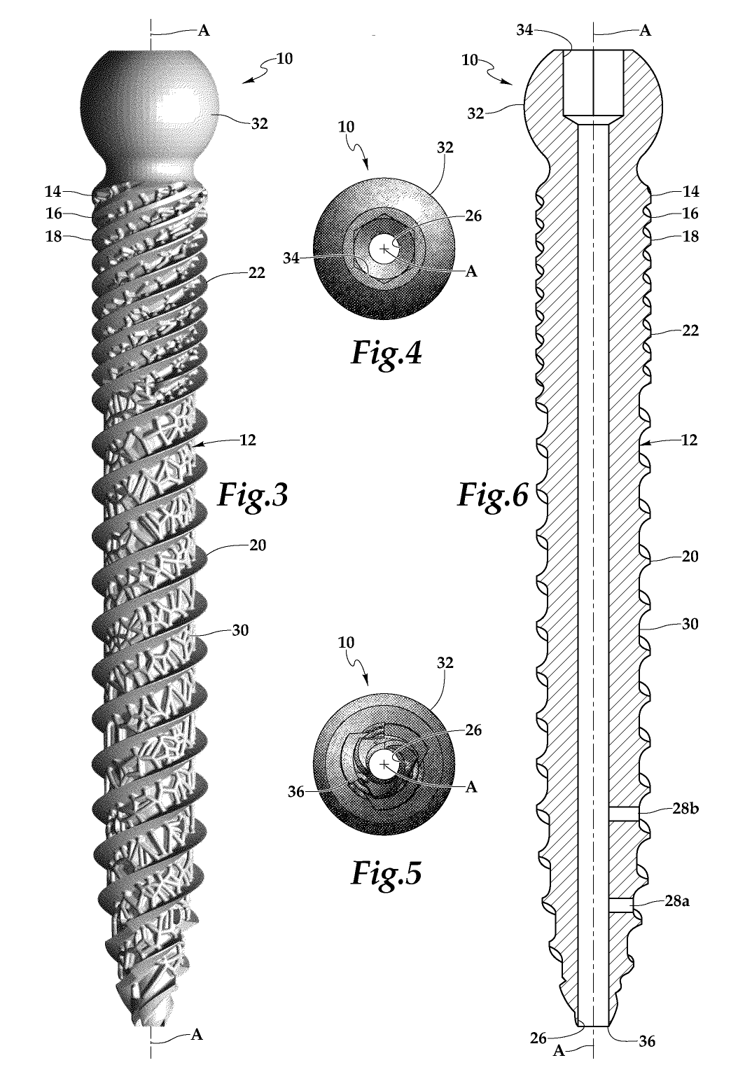

In FIG. 3, a side elevation view of bone screw 10 reveals the integration of a head 32 with the shank 12. The head 32 is integrally connected and may be deburred with edges broken to a maximum radius of around 0.005 inch, reducing the risk of tissue irritation while preserving critical dimensions. A driver-receiving element 34 (e.g., a hex socket) can be formed within the head 32, typically measuring around 3.56 ± 0.01 mm across a hex or equivalent geometry. This arrangement ensures reliable torque transfer during insertion and removal procedures, whether performed in open or minimally invasive surgical settings. From the side perspective, one may observe part of the osteo-porous lattice structure 30 close to the minor diameter of the shank 12. That lattice is integrally created during the additive build, thereby allowing a smooth gradient from denser metal to porous architecture without internal seams or bonding lines.

Referring next to FIG. 4, a proximal-end view exposes the proximal-most portion of the bone screw 10. The head 32 occupies the center of this perspective, with the driver-receiving element 34 visible from above. As discussed in other figures, surrounding the head 32, the leads of the triple threads 14, 16, 18 appear offset by 120°, confirming that each revolution advances the screw significantly. The axial bore 26 extends downward from the top surface, intended to channel various biologic or cementitious materials once the screw is implanted. Surgeons can thus deliver bone cement, growth factors, or antibiotics directly into the vertebral body via the fenestrations 28 in alignment with the bore 26, improving the chances of fusion success or augmenting fixation in compromised bone. Because the bone screw 10 is manufactured from a single-build additive method, features like the lattice structure 30 and the fenestrations 28 can be precisely located, ensuring minimal variability from design to final part.

Moving on to FIG. 5, a distal-end view showcases the distal end of the bone screw 10. From this vantage, the osteo-porous lattice structure 30 may partly wrap around the lower portion of the shank 12, especially near the minor diameter where bone ingrowth is encouraged. The fenestrations 28, including the fenestrations 28a, 28b, can also be noted, intersecting the axial bore 26. Such fenestrations 28 might be symmetrically placed or staggered, depending on design preferences. Additionally, the triple-lead threads 14, 16, 18 converge near the bottom, culminating in a region that tapers toward the distal tip 36. If employed, a self-tapping distal tip 36 intersected by a flute 38 can simplify insertion by eliminating the requirement for a separate tapping instrument. The uniform thread depth from top to bottom ensures consistent performance across the cortical and cancellous segments of bone.

In certain embodiments, each fenestration 28a, 28b opens from the axial bore 26 into an inter-thread root region 49 or valley between adjacent thread crest portions 43. The fenestrations 28 may be circumferentially positioned between adjacent ones of the first, second, and third helical threads 14, 16, 18 so that the fenestrations 28 do not intersect or substantially weaken the dense, load-bearing crest portions 44. The fenestrations 28 may open into regions occupied by or adjacent to the osteo-porous lattice structure 30, allowing injected bone cement, biologic material, antibiotic solution, or other flowable material to pass from the axial bore 26 through the fenestrations 28 and into or through the lattice structure 30 before entering surrounding bone. In one embodiment, the fenestrations 28 are located in the cancellous thread form 20 or distal cancellous segment of the shank 12, while the cortical thread form 22 or proximal cortical segment is substantially free of fenestrations. This arrangement permits flowable material to be delivered preferentially to the cancellous or trabecular region of a vertebral body while preserving an uninterrupted dense cortical-thread region for engagement with denser cortical bone.

FIG. 6 is a longitudinal cross-sectional view that further clarifies how the cancellous thread form 20 transitions into the cortical thread form 22 along the shank 12. While the cancellous segment demonstrates a more widely spaced pitch for trabecular bone, the cortical portion tightens the pitch for denser, peripheral bone. These thread forms are not disjointed; rather, they interconnect smoothly so that the three helical threads 14, 16, 18 maintain a constant thread depth while traversing each form’s flank angles (approximately 36°) and included angle (about 60°). With the offset leads advancing the screw approximately three times faster per turn, insertion time is reduced and torque is spread across multiple thread sets, mitigating localized stress. Surgeons handling osteoporotic cases may introduce bone cement or growth factors via the axial bore 26 and fenestrations 28, enhancing the bone screw’s anchorage and fusion potential. Because the entire bone screw 10 is constructed via a single-build additive manufacturing process, transitional zones between the lattice structure 30 and the denser metal matrix show no visible interface lines or bonding seams. This approach allows precise control over the region and porosity of the lattice structure 30, typically between 10% and 20%, approximating a scanned three-dimensional cancellous bone pattern. Such a scaffold fosters osseointegration and reduces the risk of loosening under cyclical spinal loads. In another embodiment, these thread forms are not disjointed; rather, they interconnect smoothly so that the three helical threads 14, 16, 18 maintain a substantially continuous thread profile while transitioning between the cancellous thread form 20 and the cortical thread form 22. In certain embodiments, the thread profile has an included thread angle of about 60° measured between opposed thread flank surfaces in an axial cross-section, while the helical path of each thread start has a lead angle or helix angle of about 36° measured at a selected pitch diameter relative to a plane perpendicular to the longitudinal axis A.

Collectively, FIGS. 1-6 illustrate a bone screw 10 in which a multi-thread profile, including cancellous and cortical thread forms 20, 22, an internal material-delivery pathway defined by the axial bore 26 and fenestrations 28, and a lattice-based scaffold provided by osteo-porous lattice structure 30 are integrated into a single, unitary implant body. In some embodiments, the bone screw 10 may undergo hot isostatic pressing after the additive build to elevate material density and fatigue strength, followed by final finishing operations. All exposed edges—including at the head 32—can be deburred to a maximum radius of 0.005 inch, aligning with best practices for implanted devices. In this manner, the bone screw 10 addresses the needs for rapid insertion, high pull-out strength, targeted biologic or cementitious delivery, and enhanced osseointegration in spine and other orthopedic applications.

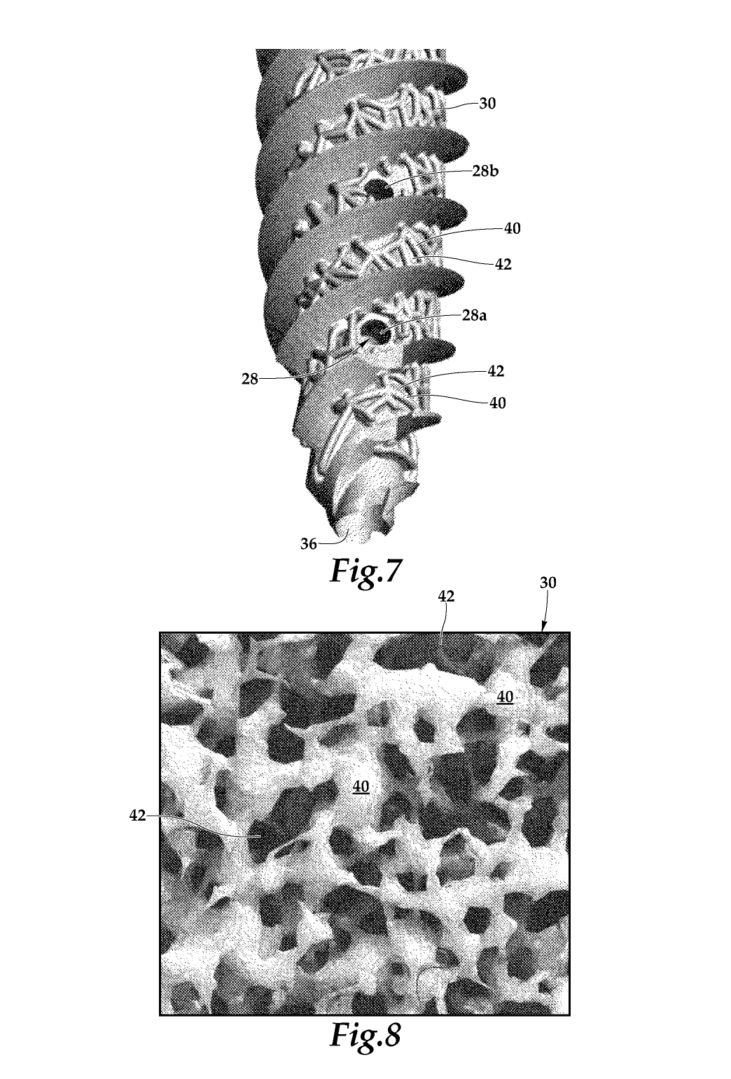

Referring now to FIG. 7, this enlarged or detailed view focuses on the osteo-porous lattice structure 30 formed along the shank 12 and the first, second, and third helical threads 14, 16, 18 of the bone screw 10. In this magnified depiction, the porous region appears as a network of interconnected struts 40, each strut 40 defining open pores 42 that collectively emulate the natural trabecular architecture of spongy bone. Unlike a surface coating, this osteo-porous lattice structure 30 is integrally built during the same additive manufacturing process that produces the denser metal portions of the bone screw 10, ensuring a continuous interface from solid metal to porous lattice. By merging these domains in a single build, the design markedly reduces the risk of delamination and bolsters the overall stability of the implant. Each strut 40 in the lattice structure 30 may transition from a more solid titanium alloy core to increasingly open, porous sections, thus avoiding abrupt changes in stiffness that can generate harmful stress risers. That is, the lattice region may transition from a more solid titanium alloy core to increasingly open porous sections. Typical pores 42 range in size from tens to hundreds of microns, an optimal dimension (e.g., a dimension suitable) for osteoblast attachment and subsequent bony integration. As the patient’s bone remodels around and through these interconnected openings, the bone screw 10 becomes biologically anchored, lowering the likelihood of loosening over time—particularly under repetitive loads inherent in spinal or orthopedic applications. Although the outer crests of the first, second, and third helical threads 14, 16, 18 remain relatively dense to preserve mechanical anchoring performance, the lattice structure 30 occupies thread flanks and interstitial areas near the minor diameter. In these zones, the struts 40 and pores 42 help distribute compressive, tensile, and shear forces more naturally into the surrounding bone, mitigating stress shielding. Consequently, healthy bone tissue is maintained, minimizing the risk of subsidence or implant failure. As used herein, a lattice structure that “approximates,” “mimics,” or is “based on” a scanned or modeled three-dimensional trabecular or cancellous bone morphology need not be an exact reproduction of a particular patient’s bone or of a particular scanned specimen. Rather, the lattice structure 30 may reproduce, adapt, scale, smooth, simplify, randomize, repeat, or otherwise approximate one or more trabecular-bone characteristics, including pore size distribution, strut thickness distribution, pore interconnectivity, local curvature, anisotropy, surface roughness, or spatial variation in porosity. The morphology may be derived from volumetric or surface data obtained by CT scanning, micro-CT scanning, laser scanning, optical scanning, or another imaging or scanning technique. The morphology may also be generated or modified using computer-aided design (CAD), lattice-generation software, topology-optimization software, stochastic or periodic unit-cell generation, mathematical modeling, or combinations of scanned data and modeled data. The source data may be patient-specific, specimen-based, library-based, averaged from multiple bone samples, or synthetically generated, provided that the resulting lattice structure 30 presents a trabecular-like three-dimensional network of struts 40 and pores 42 suitable for bone ingrowth or on-growth.

In certain embodiments, the outer crest portions 43 of the first, second, and third helical threads 14, 16, 18 are substantially fully dense titanium alloy. As used herein, “substantially fully dense” may mean at least about 95% dense, at least about 98% dense, or at least about 99% dense relative to the theoretical density of the titanium alloy, depending on the selected additive manufacturing and post-processing parameters. These dense crest portions 43 preserve thread strength, insertion durability, and pull-out resistance. The osteo-porous lattice structure 30 may be confined to, concentrated in, or preferentially disposed along the thread flank regions 45, 47, inter-thread root regions 49, and portions of the shank 12 adjacent the minor diameter. In some embodiments, the lattice structure 30 does not extend through the outermost load-bearing crest portions 43. In other embodiments, the lattice structure 30 approaches but does not interrupt the dense crest portions 44. The transition from the substantially fully dense titanium alloy regions to the osteo-porous lattice structure 30 may be a continuous integral transition formed during the same additive manufacturing build, rather than a coating, sleeve, insert, bonded member, or post-build porous layer.

Overall, this enlarged view illustrates the carefully orchestrated balance between dense and porous regions that endows the bone screw 10 with both mechanical strength and biological compatibility. The struts 40 of the osteo-porous lattice structure 30 function as scaffolding for new bone formation, yielding stronger fixation, diminished risk of loosening, and potentially more rapid bony integration compared to solid, fully dense screws. By visibly showcasing the integral nature of the porous matrix along the screw’s shaft and threads, FIG. 7 emphasizes how advanced additive manufacturing techniques—such as electron beam melting or direct metal laser sintering—enable a unified implant design that supports robust surgical instrumentation and patient healing.

Referring now to FIG. 8, another enlarged, detailed view illustrates an alternative osteo-porous lattice structure 30 formed on the bone screw 10. In this embodiment, the porous region may again be defined by interconnected struts 40 and pores 42, but the local geometry, spacing, orientation, thickness, or density of the struts 40 and pores 42 may differ from the arrangement shown in FIG. 7. In this way, the alternative architecture may provide a different balance between local stiffness and openness while still approximating a scanned three-dimensional spongy bone surface and promoting bone on-growth and bone ingrowth. As with the embodiment of FIG. 7, the alternative osteo-porous lattice structure 30 is preferably integrally produced during the same additive manufacturing build as the surrounding denser metal of the shank 12 and threads 14, 16, 18, thereby preserving a continuous interface and avoiding seams, bonded joints, or separate porous coatings.

In some embodiments, the alternative lattice structure of FIG. 8 may remain concentrated along thread flanks and interstitial regions near the minor diameter of the shank 12, while the outer crests of the first, second, and third helical threads 14, 16, 18 remain relatively dense to preserve mechanical anchoring performance. The pores 42 may again range from tens to hundreds of microns, and the overall porous region may exhibit a porosity of approximately 10%–20%. By varying the porous architecture while maintaining the integrated nature of the build, the alternative lattice structure 30 may be selected to tailor load transfer, mitigate stress shielding, encourage bony integration, and reduce the likelihood of loosening under repetitive loading. The struts 40 and pores 42 may likewise cooperate to distribute compressive, tensile, and shear forces more naturally into surrounding bone. The alternative lattice structure 30 may likewise be arranged so that dense thread crest portions 43 remain available for mechanical bone engagement while the porous regions are positioned in the flank regions 45, 47 and inter-thread root regions 49. Thus, different lattice geometries may be used without changing the overall dense-crest/porous-root architecture of the screw.

As with the embodiment of FIG. 7, the alternative osteo-porous lattice structure 30 of FIG. 8 may cooperate with the axial bore 26 and fenestrations 28 to facilitate delivery of bone cement, growth factors, antibiotics, or other biologic material into surrounding bone. Thus, FIG. 8 underscores that different porous surface topographies or lattice patterns may be implemented on the same general bone screw platform while retaining the advantages of a unitary, additively manufactured implant having triple-lead thread geometry, fenestration-based delivery capability, and enhanced osseointegration. The axial bore 26, fenestrations 28, and osteo-porous lattice structure 30 may define an interconnected delivery network. In such embodiments, the axial bore 26 provides a longitudinal delivery passage, the fenestrations 28 provide transverse outlets, and the lattice structure 30 provides a local distribution scaffold around the outlets. The lattice structure 30 may therefore receive, wick, distribute, or locally retain flowable material exiting the fenestrations 28, while also providing open pores 42 for bone ingrowth.

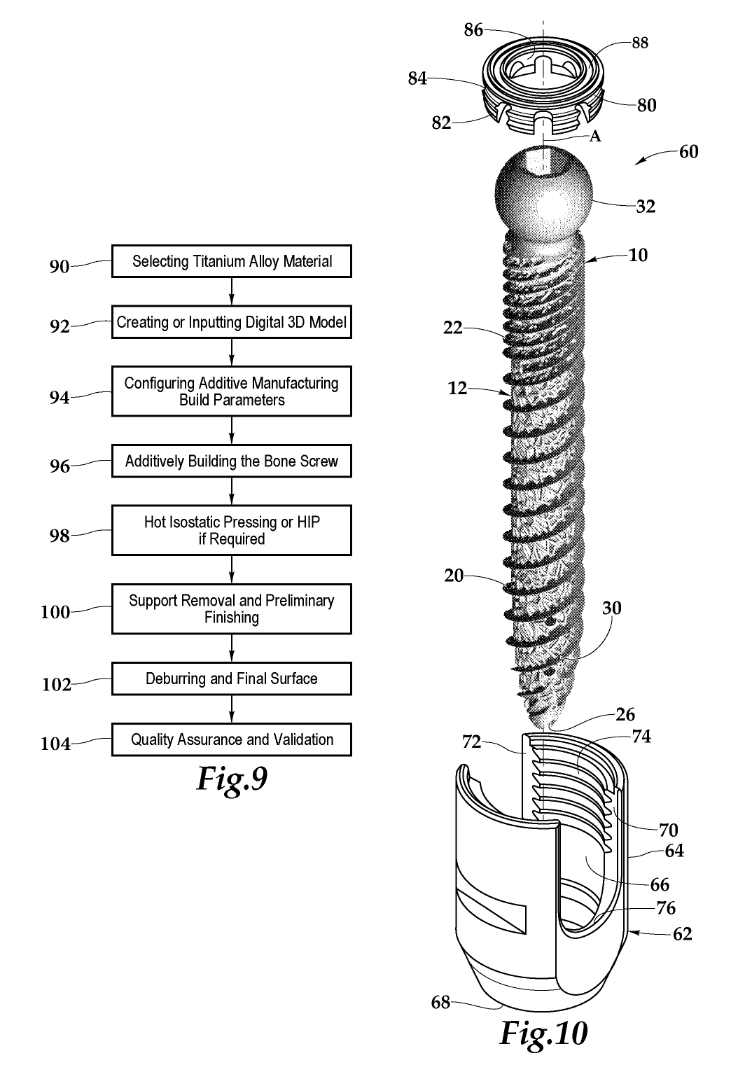

Referring now to FIG. 9, a flowchart illustrates an exemplary method of manufacturing the disclosed bone screw 10. This process describes a single-build, additive approach using a titanium alloy material conforming to stringent medical standards, culminating in a final bone screw 10 that includes the shank 12 with first, second, and third helical threads 14, 16, 18, the head 32, the axial bore 26, the fenestrations 28, and the osteo-porous lattice structure 30. The flowchart outlines the steps or “blocks” undertaken to produce a unified implant, consistent with the described triple-lead configuration, cancellous versus cortical thread forms, and lattice-based porosity.

At Block 90 (Selecting Titanium Alloy Material), the process begins by choosing a titanium alloy that meets approved medical-grade specifications. An alloy such as 6Al–4V ELI is preferred, known for its biocompatibility, corrosion resistance, and favorable mechanical properties. In many cases, the alloy must also satisfy ASTM F3001 requirements for both composition and purity to ensure safety and consistent implant performance. At this stage, raw alloy powder is typically subjected to incoming quality checks, ensuring its particle size distribution and chemical composition match the additive manufacturing (AM) system’s parameters. At Block 92 (Creating or Inputting a Digital 3D Model), next, a digital 3D model of the bone screw is either created or imported into computer-aided design (CAD) software. This model defines all the critical geometry, including the shank 12, which is subdivided into at least two segments: one featuring a cancellous thread form 20 with a more widely spaced pitch, and another featuring a cortical thread form 22 with a denser pitch. The model also captures the triple-lead configuration for threads 14, 16, 18—offset by about 120°—and sets out the axial bore 26, fenestrations 28, and the osteo-porous lattice structure 30 that exhibits approximately 10%–20% porosity. This digital design ensures each region is precisely located, including the head geometry for driver engagement, and any transitional fillets or gradient zones to mitigate abrupt material changes.

The digital 3D model may define separate build regions within a single unitary screw body. For example, the head 32, central core of the shank 12, distal tip 36, and crest portions 43 of the first, second, and third helical threads 14, 16, 18 may be assigned as dense titanium alloy regions. The thread flank regions 45, 47 inter-thread root regions 49, and regions adjacent the minor diameter of the shank 12 may be assigned as osteo-porous lattice regions having about 10% to about 20% porosity. The digital model may further position the fenestrations 28 so that each fenestration opens from the axial bore 26 into a lattice-bearing inter-thread root region 49 while avoiding intersection with the dense crest portions 43.

The osteo-porous lattice structure 30 may be generated within the digital 3D model using scanned or modeled trabecular-bone data. For example, the lattice structure 30 may be generated from CT data, micro-CT data, laser-scanning data, or optical-scanning data obtained from trabecular or cancellous bone. Alternatively, or in combination, the lattice structure 30 may be generated using CAD-defined trabecular geometry, a mathematical trabecular-bone model, stochastic lattice generation, periodic or non-periodic unit cells, topology optimization, or other software-based lattice-generation techniques. The resulting lattice structure 30 may be scaled, clipped, blended, or otherwise adapted to the thread flank regions 45, 47, inter-thread root regions 49, and regions adjacent the minor diameter of the shank 12 while preserving substantially dense thread crest portions 43.

At Block 94 (Configuring Additive Manufacturing Build Parameters), using the finalized model, an operator sets up the print job in specialized software for the chosen AM platform (e.g., electron beam melting (EBM) or direct metal laser sintering (DMLS)). The build orientation is critical. For instance, orienting the shank 12 vertically might facilitate cleaner thread formation and minimize support structures, whereas tilting or horizontally orienting the design can optimize thermal stress distribution. Support strategies for the head 32 and the tip region may also be specified. Process parameters—such as laser/electron beam power, scanning speed, hatch spacing, and layer thickness—are precisely tailored to the 6Al–4V ELI powder. Continuing with the methodology, at Block 96 (Additively Building the Bone Screw), once build parameters are locked in, the system commences the single-build process. A thin layer of titanium powder is deposited onto the build platform, then selectively fused to match the digital cross-sections of the bone screw. Layer by layer, the machine forms dense metal regions (for the thread crests, head, etc.) and porous lattice regions (for the osteo-porous lattice structure 30). Throughout this build, the first, second, and third helical threads 14, 16, 18 are generated with their leads offset by approximately 120°, ensuring the correct triple-lead configuration. Simultaneously, the axial bore 26 is formed as a continuous channel from the proximal end to the distal tip 36, and the fenestrations 28 are constructed so they intersect that bore. This integrated approach ensures that no secondary bonding or post-build assembly is required, such that the entire bone screw, including the osteo-porous lattice structure, is produced during the single additive manufacturing build.

During the additive build, the dense titanium alloy regions and the osteo-porous lattice regions are formed as integral regions of the same unitary screw body. The build may simultaneously form the substantially fully dense thread crest portions 43, the porous lattice structure 30 in the flank regions 45, 47, the root regions 49, the axial bore 26, and the fenestrations 28. The fenestrations 28 may be indexed circumferentially and axially in the build file so that they open into the porous lattice regions rather than through the load-bearing thread crest portions 43. In this manner, the axial bore 26, fenestrations 28, and lattice structure 30 are produced in the same additive manufacturing operation without requiring a post-build porous coating, bonded porous layer, or separately attached porous insert.

At Block 98 (Hot Isostatic Pressing or HIP, If Required), following additive fabrication, an optional (but often recommended) hot isostatic pressing step in accordance with ASTM F3001 may occur. This block equalizes internal stresses, closes minimal internal porosities within the dense metal, and boosts fatigue resistance. The part is typically sealed in a high-temperature, high-pressure environment for a prescribed duration. While HIP is not mandatory for every design, it is frequently performed to guarantee a robust titanium microstructure, particularly when the implant will experience significant cyclical loading in vivo. Next, at Block 100 (Support Removal and Preliminary Finishing), once the HIP cycle concludes, any residual build supports (if used) are removed. This step may involve mechanical cutting, electrical discharge machining, or abrasive blasting. Operators then inspect the raw, near-net-shape part for dimensional accuracy—particularly verifying the radial distances of the first, second, and third helical threads 14, 16, 18, the alignment of fenestrations 28 with the axial bore 26, and the correct location and porosity level for the osteo-porous lattice structure 30. If the design includes a distal tip 36 that is intersected by a flute 38 to define a self-tapping point, that geometry is also checked here. At Block 102 (Deburring and Final Surface Finish), any post-processing steps necessary to yield the final bone screw configuration are undertaken. In this block, sharp edges are deburred to a maximum radius of approximately 0.005 inch, preventing tissue irritation. Polishing or light surface blasting in compliance with recognized standards (e.g., ASTM F86) may be applied to remove loose particulate or partially sintered areas. Care is taken to preserve the 10%–20% pore fraction in the lattice structure 30 and the fidelity of the threads.

Post-processing may include, for example, removing unfused powder and loose particulate from the axial bore 26, fenestrations 28, and open pores 42 of the osteo-porous lattice structure 30. Such removal may include air flow, fluid flushing, ultrasonic cleaning, vacuum extraction, mechanical agitation, or other cleaning operations suitable for maintaining open communication between the axial bore 26, the fenestrations 28, and the lattice structure 30. Surface finishing, blasting, polishing, passivation, cleaning, and sterilization steps may be controlled so that the fenestrations 28 remain patent and the lattice structure 30 retains the intended 10% to 20% porosity.

At Block 104 (Quality Assurance and Validation), lastly, the bone screw 10 undergoes mechanical testing (tensile, torsional, pull-out tests) and dimensional checks. Quality assurance may confirm that the cancellous thread form 20 and cortical thread form 22 match the designed pitch, thread depth, included thread angle, and lead or helix angle, including, in certain embodiments, an included thread angle of about 60° and a lead or helix angle of about 36°. Radiographic or CT scans may confirm continuity in the fenestrations 28 and the internal axial bore 26, alongside verifying the open geometry of the lattice structure 30. If the device meets tolerance criteria, it is packaged and labeled for surgical use. By proceeding through Blocks 90 to 104, the manufacturing method completes all stages required by some embodiments: from selecting a suitable titanium alloy and building the entire implant—threads, head, axial bore, fenestrations, and lattice structure—in a single additive operation, through finishing and optional HIP. This integrated approach yields a bone screw 10 with a robust triple-lead geometry, seamlessly integrated porous scaffolding, and fenestrations for biologic delivery, each feature specifically tailored to enhance ease of insertion, secure bone fixation, and improved osseointegration.

Quality assurance may include verifying that the first, second, and third helical threads 14, 16, 18 remain continuous through the cancellous and cortical thread forms 20, 22 and maintain the approximately 120° phase relationship; verifying that the crest portions are substantially fully dense; verifying that the lattice structure 30 is located in the intended flank regions 45, 47 and the root regions 49; and verifying that the fenestrations 28 open from the axial bore 26 into the lattice-bearing inter-thread root regions 49. Computed tomography, radiography, borescope inspection, flow testing, dimensional inspection, or a combination thereof may be used to confirm open communication between the axial bore 26, fenestrations 28, and porous lattice structure 30.

Turning now to FIG. 10, depicted is a perspective, exploded view of the bone screw 10 (initially shown in FIG. 1) incorporated into a representative bone screw fixation system 60. In this embodiment, a rod is secured to the bone screw 10 through a tulip 62 (sometimes referred to as a “receiving member”) and additional locking components, thereby forming a stable construct suitable for spine fusion or other orthopedic procedures requiring rigid fixation. The fixation system 60 includes the tulip 62, which defines a housing 64 having a housing bore 66 at one longitudinal end and an opening 68 at the other. Aligned along a transverse axis (orthogonal to the tulip’s longitudinal axis L) are two U-shaped receiving slots 70, 72 that intersect the opening 68. These receiving slots 70, 72 are configured to accept the rod, enabling it to rest within the tulip 62 while eventually being locked in place. Internally, the housing 64 includes internal threads 74 spanning between the receiving slots 70, 72. Through the opening 68 and housing bore 66, the bone screw 10 is inserted along the longitudinal axis L of the tulip 62. The fixation system 60 may include any embodiment of the bone screw 10 described herein, including the bone screw 10 having the dense crest portions 43, osteo-porous lattice structure 30, axial bore 26, fenestrations 28, cancellous thread form 20, cortical thread form 22, and first, second, and third helical threads 14, 16, 18. The bone screw 10 may be supplied together with the tulip 62, coupling collar 80, a locking member, and the rod as a spinal fixation system or kit.

At the lower end of the housing bore 66, a spherical-shaped seat 76 is formed to accommodate the head 32 of the bone screw 10. Because the head 32 of the screw is substantially spherical, it can interface with the seat 76 to form a universal pivoting connection. In one implementation, this polyaxial linkage permits angular displacement of the tulip 62 relative to the bone screw 10, offering surgeons the flexibility to position and realign the rod as needed. The head 32 may also include a driver-receiving element 34 (for example, a hex socket) that remains accessible through the tulip 62 when adjustments are required. Once the bone screw 10 is anchored into bone, typically in a vertebral pedicle, the tulip 62 can pivot around the spherical seat 76 to capture the rod in a range of angles.

The shank 12 of the bone screw 10 emerges through the bottom side of the tulip 62 in this exploded perspective, revealing the first, second, and third helical threads 14, 16, 18 that extend from a proximal region (near the head 32) down to the distal portion. Each thread transitions from a cancellous thread form 20—having a more widely spaced pitch for spongy bone—to a cortical thread form 22—with a tighter pitch for denser bone. The leads of the three threads 14, 16, 18 remain offset by about 120°, thereby expediting insertion and enhancing mechanical purchase. Notably, an axial bore 26 runs through the shank 12, intersected by fenestrations 28 that allow targeted injection of biologics or cement. In this exploded view, one can appreciate that the entire screw 10, including its osteo-porous lattice structure 30, is produced via a single additive manufacturing build of a titanium alloy.

Situated above the head 32 of the bone screw 10 in FIG. 10 is a coupling collar 80, which is designed to interface with the substantially spherical head 32. The coupling collar 80 may include multiple resilient fingers 82 arranged circumferentially about a collar body 84. These fingers 82 may provide a snap-fit or retaining engagement with the spherical head 32 when assembled, while still permitting polyaxial movement of the tulip 62 relative to the bone screw 10 before final locking. A window 86 or aperture through the coupling collar 80 may permit continued access to the driver-receiving element 34. The coupling collar 80, rather than the spherical head 32, may present a deformable rod-engaging face 88 configured to deform against or conform to the rod when compressive force is applied by the locking member.

A driving member, which may be a set screw, may have an upper socket (e.g., a hex or Torx recess) for torque application and threads designed to mate with the internal threads 74 of the tulip 62. When the set screw is advanced through the opening 68 and engages the internal threads 74, its lower portion approaches the coupling collar 80. As the set screw is tightened, these faces clamp around the rod, increasing frictional engagement and stabilizing the rod’s position. Because the face 88 may deform slightly, the contact area along the surface of the rod is expanded, minimizing stress concentrations and improving overall mechanical lock. In some embodiments, the rod itself may be smooth, or it may include ridges, serrations, or other surface finishes to further enhance contact with the coupling collar 80 and set screw. Regardless of the rod’s texture, the principle remains: driving the set screw downward compresses the coupling collar 80 onto the spherical head 32 of the bone screw 10, simultaneously securing the rod within the receiving slots 70, 72. By virtue of the spherical seat 76, the screw 10 can pivot polyaxially, allowing the rod to be aligned at variable angles relative to the pedicle anchor point in the vertebral body. Once the desired orientation is achieved, final torque is applied to the set screw, locking the system in place.

Thus, FIG. 10 highlights how the bone screw 10 interfaces with a multi-component fixation system 60: the tulip 62, coupling collar 80, set screw, and rod. This synergy leverages the bone screw’s triple-lead threads 14, 16, 18, fenestrations 28, and the osteo-porous lattice structure 30 to provide robust attachment in a vertebral pedicle while affording surgeons a range of angle adjustments. By producing the bone screw 10 entirely via additive manufacturing, one obtains precise control over thread geometry, internal channels, and lattice features in a single piece, thereby facilitating superior fixation, biologic integration, and mechanical stability throughout the spinal construct. Although the bone screw 10 may be claimed separately, the disclosure also contemplates a spinal fixation system comprising the bone screw 10, the tulip 62 defining opposing U-shaped receiving slots 70, 72, the coupling collar 80 having resilient fingers 82 configured to engage the spherical head 32 and a deformable rod-engaging face 88, and the locking member configured to secure the rod in the tulip 62. The tulip 62, coupling collar 80, locking member, and rod may be manufactured separately from the additively manufactured bone screw 10.

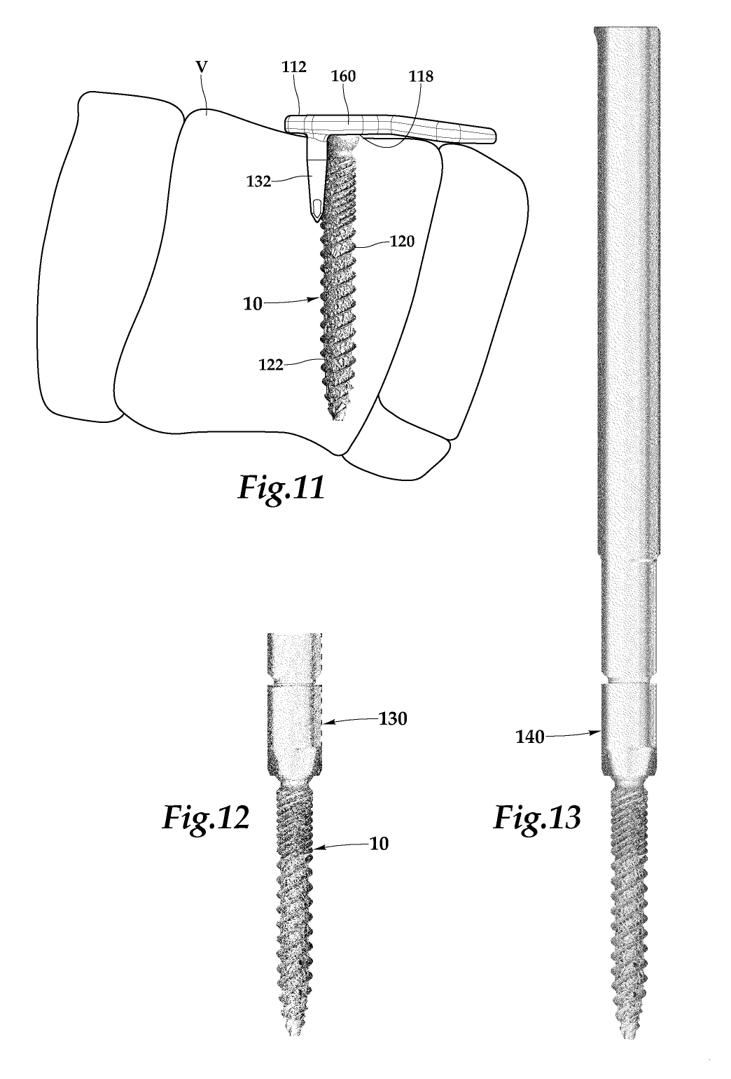

FIG. 11 presents a cross-sectional view of a portion of the bone screw 10 in conjunction with an anterior buttress staple 112, offering further insight into how these components interface to provide stable fixation. In many spine or orthopedic procedures, the anterior buttress staple 112 (alternatively referred to as a buttress plate) is used to inhibit graft or implant expulsion and to maintain alignment between adjacent bone segments—such as vertebrae V—after placement of a spinal implant I. A screw hole 118 through which the bone screw 10 passes is typically near the upper portion of the anterior buttress staple 112. The screw hole 118 may be formed with a counterbore 120 and a bore 122, ensuring that the head 32 of the screw or a portion of its shank 12 seats securely. Certain embodiments include angled surfaces that help position the screw at a desired orientation, accommodating lordosis or other anatomic angulations. A pinch point may lie between these surfaces, dictating how the anterior buttress staple 112 transitions from a region sized for the head 32 to a more tapered region for the body of the screw.

In some configurations, a pair of fangs 132 flank the screw hole 118 on the anterior buttress staple 112, aiding rotational stability by cooperating with the bone screw 10 to establish tripod-like anchorage. As the bone screw 10 is driven through the anterior buttress staple 112 and into the vertebra, the first, second, and third helical threads 14, 16, 18 engage various bone densities—cancellous or cortical—ensuring robust mechanical purchase. Meanwhile, the osteo-porous lattice structure 30 near the minor diameter fosters osseointegration, while fenestrations 28 along the axial bore 26 allow for direct injection of fusion-promoting material if needed. Because the anterior buttress staple 112 and bone screw 10 align via this screw hole 118, the cross-sectional depiction clarifies how the head 32 (or a proximal shank region) might rest against an internal contact region 160 of the staple, distributing loads across a broad surface to inhibit backout or loosening. By combining a low-profile spherical head 32 and precisely engineered thread geometry, the bone screw 10 can secure the anterior buttress staple 112 in a manner that resists both shear and distractive forces commonly experienced post-surgery. The entire arrangement underscores how the additive-manufactured geometry of the bone screw 10—featuring triple leads, fenestrations, and an osteo-porous lattice structure—interfaces with plate or staple hardware to achieve a durable, biologically integrated, and surgically efficient fixation solution.

By way of further non-limiting example, the bone screw 10 may be implemented in a variety of surgical fixation arrangements and is not limited to the fixation system 60 of FIG. 10 or the anterior buttress staple 112 of FIG. 11. For example, the bone screw 10 may be implemented with a pedicle screw fixation arrangement 130, as shown in FIG. 12, or a pedicle screw fixation arrangement 140, as shown in FIG. 13, in each of which the bone screw 10 anchors into a vertebral pedicle and cooperates with a tulip, coupling member, rod, crosslink, connector, or other spinal fixation component to stabilize one or more vertebral motion segments.

The order of execution or performance of the methods and manufacturing steps illustrated and described herein is not essential, unless otherwise specified. In other words, elements of the methods and data flows may be performed in any order, unless otherwise specified, and the methods may include more or fewer elements than those disclosed herein. By way of example, it is contemplated that executing or performing a particular element before, contemporaneously with, or after another element are all possible sequences of execution, provided that the intended result of each method step remains achievable.

While this invention has been described with reference to illustrative embodiments, the description is not intended to be construed in a limiting sense. Various modifications, substitutions, omissions, and combinations of the illustrative embodiments—as well as other embodiments of the invention—will be apparent to those skilled in the art upon reference to the present disclosure. It is, therefore, intended that the appended claims encompass any such modifications, embodiments, and equivalents thereof that fall within the scope and spirit of the inventive concepts presented herein.

What is claimed is:

1. An implantable bone screw comprising:
a shank, including first, second, and third helical threads, each of the first, second, and third helical threads extending through a cancellous thread form and a cortical thread form along respective segments of the shank, leads of the first, second, and third helical threads being offset by approximately 120° relative to one another to form a triple-lead configuration, thereby expediting insertion while maintaining secure fixation, the cancellous thread form having a more widely spaced pitch than the cortical thread form;
a head, integrally connected with the shank, the head configured for engagement with a driver;
an axial bore extending from a proximal end of the bone screw to a distal end;
at least two fenestrations located along a portion of the shank, each fenestration intersecting the axial bore to facilitate passage of biologic or cementitious materials;
an osteo-porous lattice structure disposed on at least a region of the shank, the osteo-porous lattice structure having a porosity between about 10% and about 20%, the osteo-porous lattice structure approximating a scanned or modeled three-dimensional trabecular or cancellous bone morphology, thereby promoting bone ingrowth while maintaining mechanical strength; and
wherein the entire bone screw, including the osteo-porous lattice structure, is produced via an additive manufacturing process of a titanium alloy in a single build;
wherein each of the first, second, and third helical threads is a continuous thread start extending through both the cancellous thread form and the cortical thread form while maintaining the approximately 120° phase offset;
wherein crest portions of the first, second, and third helical threads are substantially fully dense titanium alloy;
wherein the osteo-porous lattice structure is confined to thread flank regions and inter-thread root regions adjacent a minor diameter of the shank, and is integrally joined to the dense titanium alloy in a continuous dense-to-porous transition formed during the single additive manufacturing build.

2. The bone screw of claim 1, wherein said titanium alloy is 6Al–4V ELI, and wherein the additive manufacturing process is selected from electron beam melting or direct metal laser sintering.

3. The bone screw of claim 1, wherein each of the cancellous thread form and the cortical thread form has an included thread angle of about 60° measured in an axial cross-section between opposed flank surfaces, and wherein each of the first, second, and third helical threads has a lead angle or helix angle of about 36°
measured at a selected pitch diameter relative to a plane perpendicular to a longitudinal axis of the shank.

4. The bone screw of claim 1, wherein a major pitch dimension for the cancellous thread form is approximately 2.67 mm (0.105 in.) and a minor pitch dimension is approximately 1.34 mm (0.053 in.), with thread thickness between about 0.26 mm (0.010 in.) and 0.18 mm (0.007 in.), such that the triple-lead configuration advances the screw faster than a single-lead design.

5. The bone screw of claim 1, wherein all exposed edges are deburred and broken to a maximum radius of 0.005 inch, and a final surface finish is applied.

6. The bone screw of claim 1, wherein the head includes a driver interface having a nominal dimension of about 3.56 ± 0.01 mm across a hex or equivalent geometry.

7. The bone screw of claim 1, wherein the osteo-porous lattice structure is disposed along thread flanks and near the minor diameter, creating a gradient zone that mitigates stress shielding while allowing bone to infiltrate the porous matrix.

8. The bone screw of claim 1, wherein the axial bore and said fenestrations are configured to deliver a flowable material selected from the group consisting of bone cement, growth factors, or antibiotic solutions, thereby enhancing fixation in osteoporotic bone or aiding in fusion.

9. The bone screw of claim 1, wherein the triple-lead configuration formed by the first, second, and third helical threads advances approximately three times faster per revolution compared to a single-lead design, reducing insertion torque and procedure time.

10. The bone screw of claim 1, wherein the entire screw, after the additive build, is subjected to hot isostatic pressing under conditions selected to reduce internal porosity in substantially fully dense titanium alloy regions and improve fatigue strength while preserving open pores of the osteo-porous lattice structure.

11. The bone screw of claim 1, further comprising a tip integrally connected to a distal portion of the shank, the tip intersected by a flute to define a self-tapping point.

12. An implantable bone screw comprising:
a shank, including first, second, and third helical threads, each of the first, second, and third helical threads extending through a cancellous thread form and a cortical thread form along respective segments of the shank, leads of the first, second, and third helical threads being offset by approximately 120° relative to one another to form a triple-lead configuration, thereby expediting insertion while maintaining secure fixation, the cancellous thread form having a more widely spaced pitch than the cortical thread form;
a head, integrally connected with the shank, the head configured for engagement with a driver;
an axial bore extending from a proximal end of the bone screw to a distal end;
at least two fenestrations located along a portion of the shank, each fenestration intersecting the axial bore to facilitate passage of biologic or cementitious materials;
an osteo-porous lattice structure disposed on at least a region of the shank, the osteo-porous lattice structure having a porosity between about 10% and about 20%, the osteo-porous lattice structure approximating a scanned three-dimensional spongy bone surface, thereby promoting bone ingrowth while maintaining mechanical strength; and
wherein the entire bone screw, including the osteo-porous lattice structure, is produced via an additive manufacturing process of a titanium alloy in a single build;
wherein each of the first, second, and third helical threads is a continuous thread start extending through both the cancellous thread form and the cortical thread form while maintaining the approximately 120° phase offset;
wherein crest portions of the first, second, and third helical threads are substantially fully dense titanium alloy;
wherein the osteo-porous lattice structure is confined to thread flank regions and inter-thread root regions adjacent a minor diameter of the shank, and is integrally joined to the dense titanium alloy in a continuous dense-to-porous transition formed during the single additive manufacturing build;
wherein the at least two fenestrations open from the axial bore into inter-thread root regions occupied by the osteo-porous lattice structure, the fenestrations being circumferentially positioned between adjacent ones of the first, second, and third helical threads;
wherein the fenestrations are located in the cancellous-thread segment and the cortical-thread segment remains substantially free of fenestrations.

13. The bone screw of claim 12, wherein the additive manufacturing process is selected from electron beam melting or direct metal laser sintering, and wherein the titanium alloy is 6Al–4V ELI, the bone screw thereafter being subjected to hot isostatic pressing.

14. The bone screw of claim 12, wherein each of the cancellous thread form and the cortical thread form has an included thread angle of about 60° measured in an axial cross-section between opposed flank surfaces, and wherein each of the first, second, and third helical threads has a lead angle or helix angle of about 36° measured at a selected pitch diameter relative to a plane perpendicular to a longitudinal axis of the shank.

15. The bone screw of claim 12, wherein all exposed edges on the screw are deburred and broken to a maximum radius of 0.005 inch, and the bone screw is finished.

16. The bone screw of claim 12, wherein the cancellous thread form has a major pitch dimension of approximately 2.67 mm (0.105 in.) and a minor pitch dimension of approximately 1.34 mm (0.053 in.), with a thread thickness of about 0.26 mm (0.010 in.) to 0.18 mm (0.007 in.), and wherein the triple-lead configuration advances the screw approximately three times faster per revolution than a single-lead design.

17. The bone screw of claim 12, further comprising a tip integrally connected to a distal portion of the shank, the tip intersected by a flute to define a self-tapping point.

18. A method of manufacturing an implantable bone screw, comprising:
generating a digital model of an implantable bone screw, the digital model defining a shank, a head, an axial bore, at least two fenestrations, an osteo-porous lattice structure, and first, second, and third helical threads, each of the first, second, and third helical threads being defined as a continuous thread start extending through a cancellous thread form and a cortical thread form while maintaining an approximately 120° circumferential phase offset relative to the other thread starts;
assigning, in the digital model, crest portions of the first, second, and third helical threads to substantially dense titanium alloy build regions and assigning thread flank regions and inter-thread root regions adjacent a minor diameter of the shank to porous lattice build regions having a porosity between about 10% and about 20%;
generating the osteo-porous lattice structure in the digital model with a three-dimensional trabecular or cancellous bone morphology based on at least one of CT data, micro-CT data, laser-scanning data, CAD-defined trabecular geometry, or a mathematical trabecular-bone model;
selecting a titanium alloy material conforming to an approved medical grade;
additively building, in a single build from the titanium alloy material, the implantable bone screw according to the digital model, wherein the additive build forms:
the shank having the first, second, and third helical threads;
the head integrally connected with the shank and configured for driver engagement;
the axial bore extending from a proximal end of the bone screw to a distal end;
the at least two fenestrations along the shank, each fenestration intersecting the axial bore; and
the osteo-porous lattice structure in the porous lattice build regions;
wherein the at least two fenestrations are formed to open from the axial bore into inter-thread root regions occupied by or adjacent to the osteo-porous lattice
structure; and
post-processing the additively built bone screw to yield a final bone screw
configuration while maintaining open communication between the axial bore, the
fenestrations, and the osteo-porous lattice structure.

19. The method of claim 18, further comprising the step of hot isostatic pressing the additively built bone screw in accordance with ASTM F3001, wherein the titanium alloy is 6Al–4V ELI, and said additive process is selected from electron beam melting or direct metal laser sintering.

20. The method of claim 18, further comprising forming a tip on a distal portion of the shank such that the tip is intersected by a flute to define a self-tapping point, thereby allowing the bone screw to be inserted into bone without a separate tapping step.
